(19) United States
(12) Patent Application Publication
(10) Pub. No.: US 2003/0153888 A1
(43) Pub. Date: Aug. 14, 2003

(54) Three-layered pantiliner

(76) Inventor: Karen Yvonne Johnston, Pocatello, ID (US)

Correspondence Address: Dr. Karen Johnston, 6375 S. Bannock Hwy., Pocatello, ID, 83204, US

(21) Appl. No.: 10/066,449

(22) Filed: Feb. 12, 2002

(51) Int. Cl. A61F 13/15

(52) U.S. Cl. 604/378; 604/385.03

(57) ABSTRACT

My invention is a three-layered pantiliner with separate, removable and disposable layers which can be removed as needed ensuring continued protection.

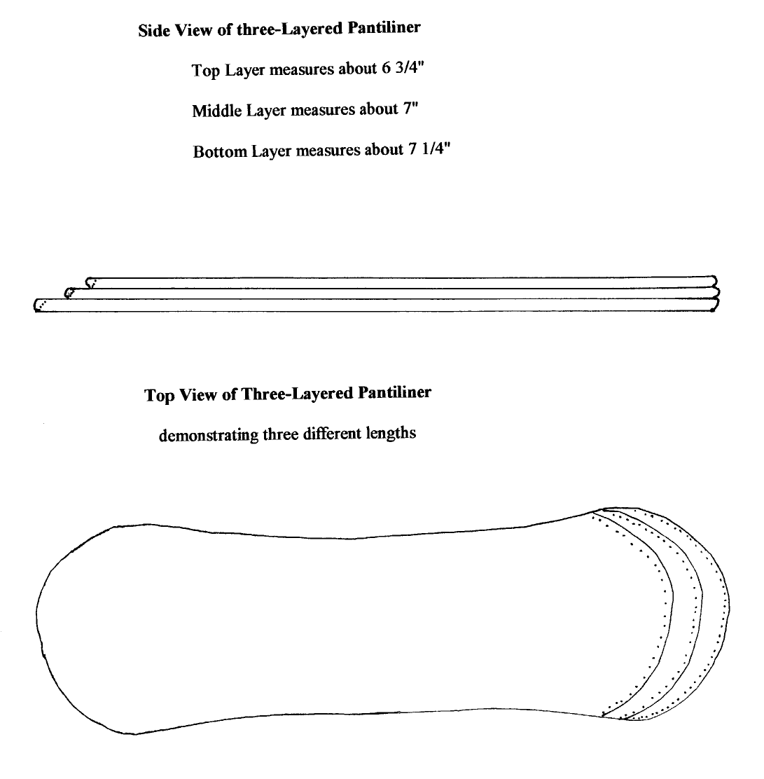

The longer version of the pantiliners has a bottom (third) layer that measures 7¼″, the middle (second) layer measures 7″, and the top (first) layer measures 6¾″, approximately. The shorter version has similar measurements beginning with a 5¾″ bottom layer.

By having three layers, rather than the customary one layer, the wearer is able to remove the top layer as needed, leaving another layer underneath, and still another layer under that, to continue the protection of her clothing.

### BACKGROUND OF THE INVENTION

Many American women wear pantiliners because they desire continual protection throughout the day. It is often inconvenient to carry extra pantiliners around to replace the one currently being worn. I created a three-layered pantiliner to wear when I go to work so I can discreetly remove the used top pantiliner and still have a pantiliner remain on my underclothing. Three layers works perfectly for a fuill day of work.

### BRIEF DESCRIPTION OF THE DRAWINGS

The attached drawing shows the three-layered pantiliner with three separate liners adhered together to form one pantiliner. The adhesive is light and does not harm the cotton on the liner underneath. The bottom liner has a slightly stronger adhesive than does the upper two layers for it adhered to the fabric of the underclothing and is a foundation for the other two layers.

The bottom (third layer) measures about 7¼″, the middle (second layer) measures about 7″, and the top (first layer) measures about 6¾″ to enable the user to easily separate one layer from another. (A shorter version will have a length of approximately 5¾″ for the bottom layer, 5½″ for the middle layer, and 5¼″ for the top layer.)

The attached drawing shows a side view and the top view of the invention.

### DETAILED DESCRIPTION OF THE INVENTION

The invention is a three-layered pantiliner with separate removable liners that can be removed throughout the day as needed, thereby ensuring continued cleanliness.

Because there are three layers, the wearer is able to remove one layer at a time as needed, leaving another layer underneath to continue the protection of her clothing.

The need for such an invention exists because the standards of personal cleanliness continue to improve. Women fear offending others by smelling unclean; therefore, they have used pantiliners for many years to insure cleanliness of their clothing. The same women who routinely use the existing pantiliners will be enthusiastic and faithful consumers of this product because it is an improvement of the existing pantiliners and will eliminate the hassle and embarrassment of carrying extra pantiliners to the office or workplace. Once consumers are familiar with the product, its convenience will make it highly marketable and it will become an everyday part of their lives.

I claim as my invention a three-layered pantiliner. I request that the material be received under the Disclosure Document Program.

1. This three-layered pantiliner is an improvement on the traditional pantiliner that has a thin cotton pad with plastic and adhesive attached to the underneath and is attached to women's underclothing for cleanliness. This invention has three layers attached to each other all worn at the same time. The bottom pad is topped with a slightly smaller pad that is topped with a still slightly smaller pad.
This three-layered concept enables the user to dispose of the used layer by peeling it off and discarding it, leaving two more clean layers to ensure continued cleanliness throughout the day.
